(19) United States
(12) Patent Application Publication
(10) Pub. No.: US 2016/0281094 A1
(43) Pub. Date: Sep. 29, 2016

(54) TECHNOLOGIES FOR MANUFACTURING AN ENGINEERED BIO-SYSTEM

(71) Applicants: John C. Weast, Portland, OR (US); Brian D. Johnson, Portland, OR (US); Tobias M. Kohlenberg, Portland, OR (US)

(72) Inventors: John C. Weast, Portland, OR (US); Brian D. Johnson, Portland, OR (US); Tobias M. Kohlenberg, Portland, OR (US)

(21) Appl. No.: 14/672,100

(22) Filed: Mar. 28, 2015

(51) Int. Cl. C12N 15/63; G01N 33/50; C12N 15/10

(52) U.S. Cl. CPC C12N 15/63; C12N 15/1079; G01N 33/5038

(57) ABSTRACT

Technologies for manufacturing an engineered biological system include determining a plurality of functions to be performed by the engineered biological system while in a corresponding state. The engineered biological system is to transition between states based on the presence of a corresponding transition trigger defined by a biological key associated with each state. A state machine mapping is generated for the manufacture of the engineered biological system. The engineered biological system is verified and subsequently activated in a host. An engineered biological system and associated method for performing a biological function are also disclosed.

### BACKGROUND

Naturally occurring biological systems may take many different forms and perform various functions. Such biological systems may additionally differ in scale, from micro-systems, such as cells, viruses, and the like, to macro-systems, which may include complex networks of micro-scale biological entities and systems. Naturally occurring biological systems offer limited methodologies for external control of the behavior of the system. However, efforts to identify and categorize the various functions and attributes of naturally occurring biological systems have resulted in repositories of various registries and databases (e.g., virus registries, genome databases, protein databases, etc.).

Biological systems may also be manufactured, resulting in a non-naturally occurring biological system. Typical manufactured biological systems are often designed to perform a particular function (e.g., a vaccine to combat a virus). Such functions tend to be focused and often unitary. Additionally, typical manufactured biological systems are limited in their adaptability to new functionality once activated in a host.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of an engineered biological system;

FIG. 2 is a simplified block diagram of at least one embodiment of a state machine that may be employed by the engineered biological system of FIG. 1;

FIG. 3 is a simplified block diagram of at least one additional embodiment of a state machine that may be employed by the engineered biological system of FIG. 1;

FIG. 4 is a simplified flow diagram of at least one embodiment of a method for manufacturing an engineered biological system; and

FIG. 5 is a simplified flow diagram of at least one embodiment of a method for designing an engineered biological system.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); (A and C); or (A, B, and C). Similarly, items listed in the form of “at least one of A, B, or C” can mean (A); (B); (C): (A and B); (B and C); (A or C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

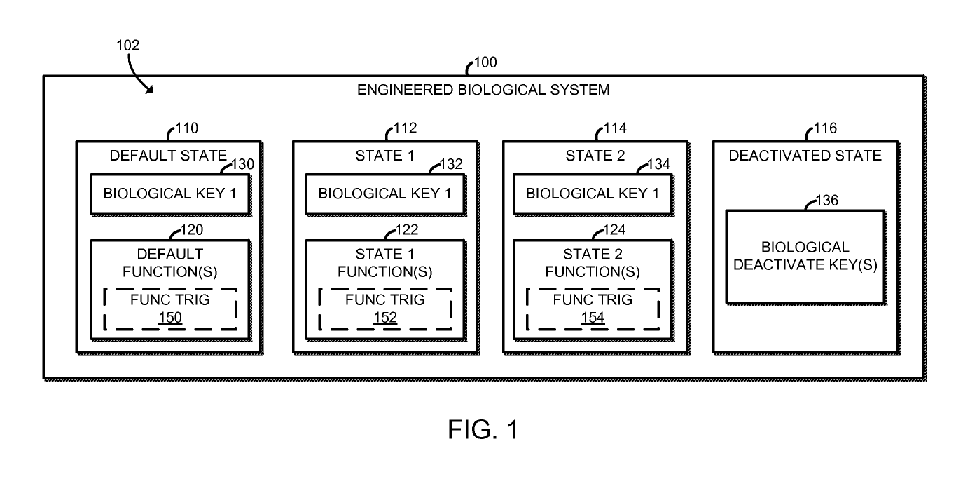

Referring now to FIG. 1, an illustrative engineered biological system 100 is configured to transition between various states and perform one or more functions (e.g., biological functions) in each state. Each state of the engineered biological system 100 is defined by an associated biological key, which defines a transition trigger that causes the engineered biological system 100 to transition to the corresponding state. For example, as shown in FIG. 1, the illustrative engineered biological system 100 includes a default state 110, a first functional state 112, a second functional state 114, and a deactivated state 116. The default state 110 defines the initial state of the engineered biological system 100 when system 100 is first associated with a target host. In the default state 110, the engineered biological system 100 may perform one or more default functions 120 in some embodiments. The deactivated state 116 defines the final or “dead” state of the engineered biological system 100, which is typically entered upon completion of the desired functionality of the engineered biological system 100. The functional states 112, 114 defines the various states of the engineered biological system 100 in which the system 100 performs one or more functions. For example, while in the first functional state 112, the engineered biological system 100 is configured to perform a corresponding state function 122. Similarly, while in the second functional state 114, the engineered biological system 100 is configured to perform a corresponding state function 124. Of course, as discussed in more detail below, the engineered biological system 100 may be designed to have additional or fewer states, and associated functions, in other embodiments depending on the desired functionality of the engineered biological system 100, characteristics of the host into which the engineered biological system 100 will be active, and/or other criteria.

Each state of the engineered biological system 100 is defined by one or more corresponding biological keys. As discussed above, each biological key defines the conditions (i.e., a transition trigger) that must be present for the engineered biological system 100 to transition to the corresponding state. In the illustrative example of FIG. 1, the first functional state 112 is defined by a biological key 132 and the second functional state 114 is defined by a biological key 134. As such, the biological keys 132, 134 define the particular stimulus or trigger condition that must be present (or not present) to cause the engineered biological system 100 to transition to the corresponding state 112, 114. For example, the presence of a particular chemical in proximity to the engineered biological system 100 may cause the system 100 to transition from the default state 110 to the first functional state 112. In that example, the presence of an a sensed temperature above a threshold amount may cause the system 100 to transition from the first functional state 112 to the second functional state 114. Further, in the present example, physical contact with a particular organ of the host by the engineered biological system 100 may cause the engineered biological system to transition from the second functional state 114 to the deactivated state 116, and so forth.

Each biological key may be embodied as any type of trigger condition that can be sensed by the engineered biological system 100. For example, each biological key may be embodied as an energetic key, an organic key, a chemical key, an external key, a contextual key, a proximity key, or any other type of biological key capable of defining a particular trigger condition. Energetic biological keys are desired to cause the engineered biological system 100 to transition states in response to a corresponding energetic transition trigger. Organic biological keys are desired to cause the engineered biological system 100 to transition states in response to an organic transition trigger. Chemical biological keys are designed to cause the engineered biological system 100 to transition states in response to a chemical transition trigger. External biological keys are desired to cause the engineered biological system 100 to transition states in response to a transition trigger external from the host. Contextual biological keys are designed to cause the engineered biological system 100 to transition states in response to a contextual transition trigger indicative of a context of the engineered biological system 100. Proximity biological keys are designed to cause the engineered biological system 100 to transition states in response to a proximity transition trigger indicative of the engineered biological system 100 being in proximity to a target system, chemical, or other biological entity (e.g., a bio-logical geo-fence).

Some states may have multiple biological keys associated with it. For example, the default state 110 and/or the deactivated state 116 may each have multiple biological keys that define different transition triggers that may cause the engineered biological system 100 to transition back to the default state 110 or the deactivated state 116. For example, the particular transition trigger required to be present to cause a state transition to the default state 110 or the deactivated state 116 may depend on the current state of the engineered biological system 100. That is, if the engineered biological system 100 is in the first functional state 112, a different transition trigger may be required to transition to the deactivated state 116 than when the engineered biological system 100 is in the second functional state 114.

As discussed above, the engineered biological system 100 is configured to perform one or more functions in some of the various states. For example, while in the first functional state 112, the engineered biological system 100 is configured to perform a first state function 122. Additionally, while in the second functional state 114, the engineered biological system 100 is configured to perform a second state function 124. In some embodiments, the engineered biological system 100 may also be configured to perform one or more functions 120 while in the default state 110. The functions 120, 122, 124 performed by the engineered biological system 100 may be embodied as any type of function capable of being performed by a biological system. For example, the engineered biological system 100 may be configured to sense for the presence of particular stimulus (e.g., biological, chemical, or energetic stimuli), replicate, express particular genes, produce particular chemical or biological matter, and/or other biological, chemical, or energetic function.

One or more of the functions performed by the engineered biological system 100 may be dependent upon a corresponding functional trigger. For example, the first state function 122 of the first functional state 112 may require the presence of a functional trigger 152. That is, the engineered biological system 100 may be configured to perform the first state function 122 only in response to the functional trigger 152. Conversely, the engineered biological system 100 may be configured to perform the second state function 124 while in the second functional state 114 regardless of any trigger condition (or, as shown in FIG. 1, may also require the presence of a corresponding functional trigger 154). The various functional triggers may be embodied as any type of trigger condition that can be sensed by the engineered biological system 100. For example, each functional trigger may be embodied as an energetic functional trigger, an organic functional trigger, a chemical functional trigger, an external functional trigger, a contextual functional trigger, a proximity functional trigger, or any other type of trigger condition.

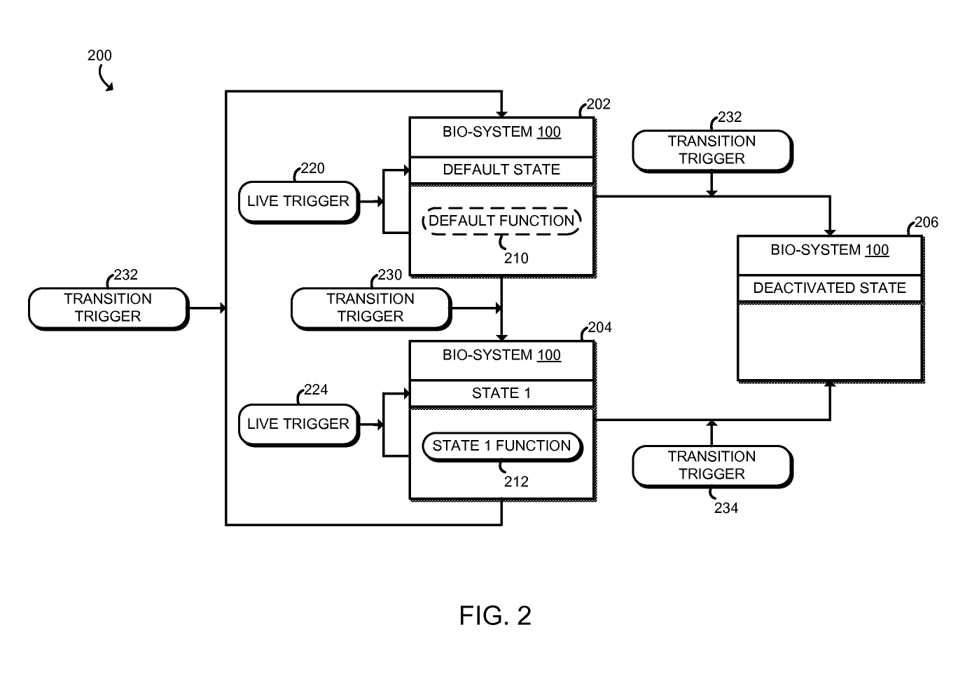

Referring now to FIG. 2, a state machine 200 of an illustrative engineered biological system 100 is shown. The state machine 200 defines the various states, transitions, and associated functions employed by the corresponding engineered biological system 100. In the illustrative embodiment, the state machine 200 includes a default state 202, a functional state 204, and a deactivated state 206. As discussed above, the engineered biological system 100 begins in the default state 202 when activated in a host. The default state 202 may or may not include default functions 210 performed by the engineered biological system 100 while in the default state 202. In some embodiments, the engineered biological system 100 may be configured to remain in the default state 202 based on a live trigger 220. The live trigger 220, and similar live triggers discussed below, is similar to the transition triggers described above and may define conditions that must be present (or not present) for the engineered biological system 100 to remain in the associated state. For example, each live trigger may be embodied as an energetic live trigger, an organic live trigger, a chemical live trigger, an external live trigger, a contextual live trigger, a proximity live trigger, or any other type of trigger condition.

While in the default state 202, the engineered biological system 100 may transition to one or more states in response to a corresponding transition trigger. For example, in the illustrative embodiment, the engineered biological system 100 may transition from the default state 202 to a first functional state 204 in response to a transition trigger 230. Alternatively, while in the default state 202, the engineered biological system 100 may transition to the deactivated state 206 in response to a transition trigger 232.

While in the first functional state 204, the engineered biological system 100 may perform one or more state functions 212. As discussed above, the engineered biological system 100 may perform the one or more state functions 212 continually or periodically. Alternatively or additionally, the engineered biological system 100 may perform one or more of the state functions 212 in response to a functional trigger as discussed above.

The engineered biological system 100 may remain in the first functional state 204 while a corresponding live trigger 224 is present. Alternatively, the engineered biological system 100 may transition back to the default state 202 in response to a transition trigger 232 or to the deactivated state 206 in response to a transition trigger 234.

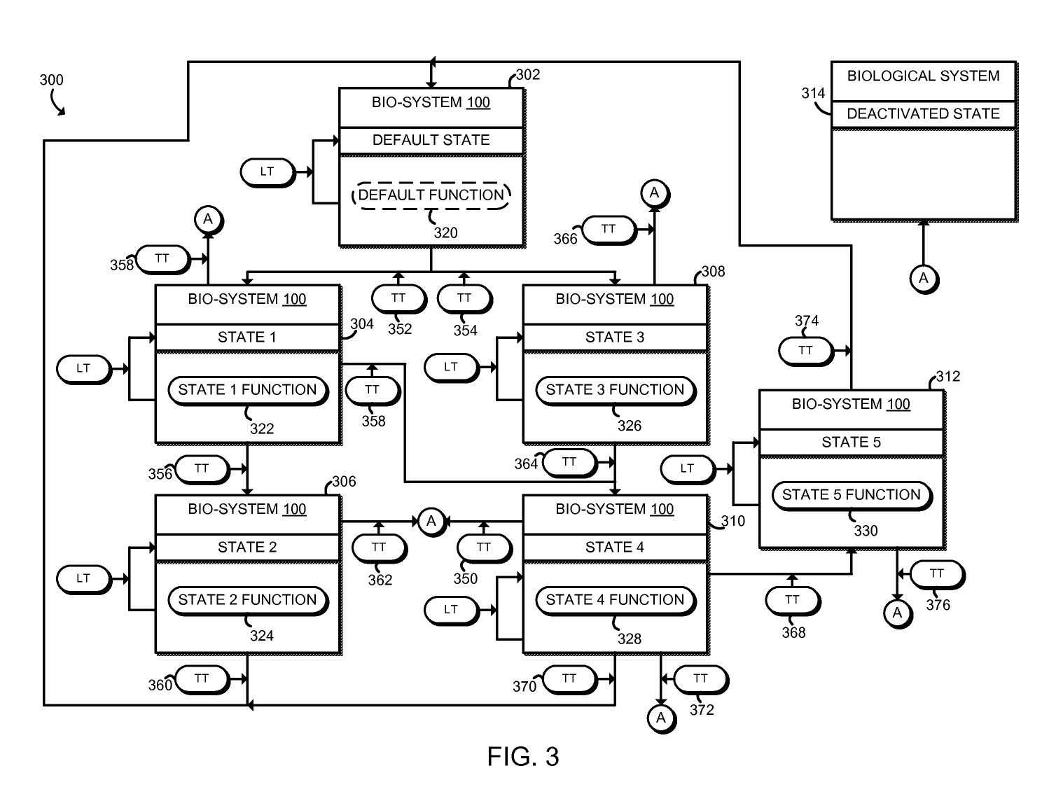

Of course, the engineered biological system 100 may be designed to employ more complicated state machines, having a larger number of states and performing additional and/or diverse functions. A more complex state machine 300, which may be implemented by the engineered biological system 100, is shown in FIG. 3. In the illustrative embodiment, the state machine 300 includes a default state 302, a first functional state 304, a second functional state 306, a third functional state 308, a fourth functional state 310, a fifth functional state 312, and a deactivated state 314. As discussed above, the engineered biological system 100 is configured to transition between the various states in response to corresponding transition triggers and perform various functions in each functional state. For example, the engineered biological system 100 begins in the default state 302 and may transition therefrom to the first functional state 304 in response to a transition trigger 350 or to the third functional state 308 in response to a transition trigger 352. The engineered biological system 100 may perform one or more state functions 322 while in the first functional state 304 and one or more state functions 326 while in the third functional state 308.

While in the first functional state 304, the engineered biological system 100 may transition to second functional state 306 in response to a transition trigger 354, to the fourth functional state in response to a transition trigger 356, or to the deactivated state 314 in response to a transition trigger 358. The engineered biological system 100 may perform one or more state functions 324 while in the second functional state 306 and one or more state functions 328 while in the fourth functional state.

While in the second functional state 306, the engineered biological system 100 may transition to default state 302 in response to a transition trigger 360 or to the deactivated state 314 in response to a transition trigger 362. Additionally, while in the third functional state 308, the engineered biological system 100 may transition to the fourth functional state 310 in response to a transition trigger 364 or to the deactivated state 314 in response to a transition trigger 366. Further, while in the fourth functional state 310, the engineered biological system 100 may transitions to the fifth functional state 312 in response to a transition trigger 368, to the default state 302 in response to a transition trigger 370, or to the deactivated state 314 in response to a transition trigger 372.

While in the fifth functional state 312, the engineered biological system 100 may perform one or more state functions 330. Additionally, while in the fifth functional state 312, the engineered biological system 100 may transition to the default state 302 in response to a transition trigger 374 or to the deactivated state 314 in response to a transition trigger 376.

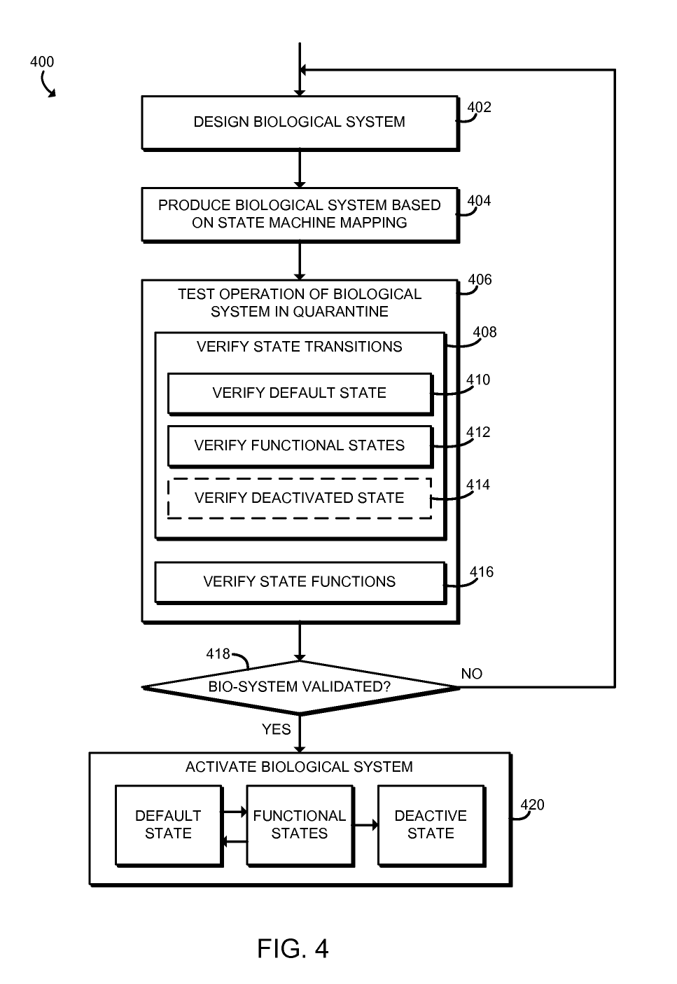

Referring now to FIG. 4, a method 400 may be implemented to manufacture an engineered biological system 100 described above. In some embodiments, portions of the method 400 may be executed by or otherwise implemented on or with the help of a computing system, such as a special-purpose biological system fabrication system.

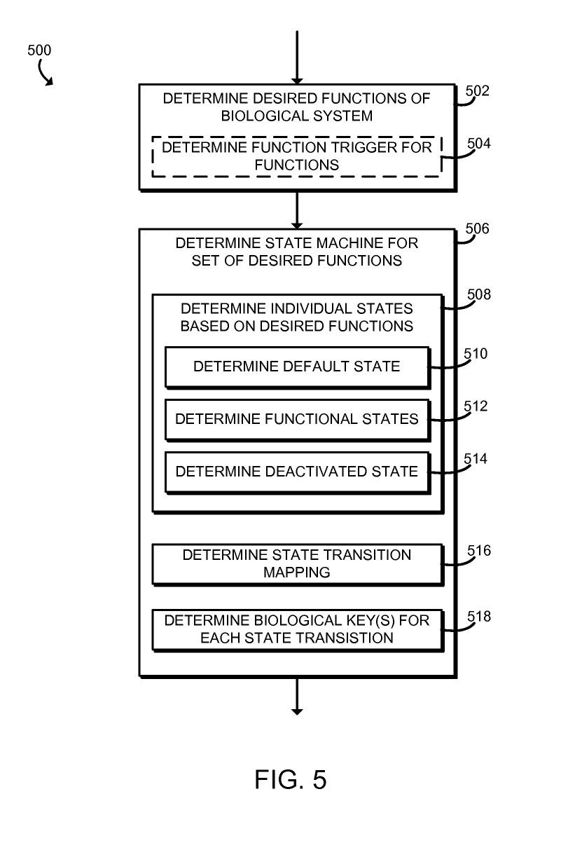

The method 400 begins with block 402 in which the engineered biological system 100 is designed. That is, in block 402, the various states, associated functions, and biological keys are determined, which may be used to generate a state machine mapping for the engineered biological system 100. To do so, as shown in FIG. 5, a method 500 for designing an engineered biological system 100 may be implemented. The method 500 begins with block 502 in which the desired functions of the engineered biological system are determined. As discussed above, the functions to be performed by the engineered biological system 100 may be embodied as any type of function capable of being performed by a biological system including, but not limited to, sensing for the presence of a particular stimulus, replicating, expressing particular genes, producing particular chemical or biological matter, and/or other biological, chemical, or energetic function. In some embodiments, one or more functional triggers may be determined in block 504 for one or more of the functions determined in block 502. Again, as discussed above, the functional triggers may be embodied as any type of trigger condition that can be sensed by the engineered biological system 100. For example, each functional trigger may be embodied as an energetic functional trigger, an organic functional trigger, a chemical functional trigger, an external functional trigger, a contextual functional trigger, a proximity functional trigger, or any other type of trigger condition.

After the desired functionality of the engineered biological system 100 is determined in block 502, the method 500 advances to block 506 in which the state machine for the set of desired functions is determined. To do so, in block 508, the individual states of the state machine of the engineered biological system 100 are determined based on the desired functions. For example, a default state is determined in block 510, one or more functional states are determined in block 512, and a deactivated state is determined in block 514. Additionally, the state transition mapping is determined in block 516. That is, the various transitions between the individual states is determined in block 516 (e.g., how the engineered biological system 100 paths between the various states). Subsequently, in block 518, the biological keys for each state transition (i.e., the biological key(s) associated with each state) are determined. That is, the various trigger conditions that cause the engineered biological system to transition from one state to another are determined in block 518. In this way, the state machine mapping of an engineered biological system 100 may be determined based on the desired functionality and trigger conditions.

Referring back to FIG. 4, after the engineered biological system has been designed in block 402, the engineered biological system is produced in block 404 based on the state transition mapping determined in block 402. After the engineered biological system 100 is produced in block 404, the engineered biological system 100 is tested in a quarantined environment in block 406. To do so, the engineered biological system 100 may be tested in an experimental host or tested in a laboratory or other controlled environment. In block 406, the behavior and functionality of the engineered biological system 100 is verified. For example, in block 408, the various transition of the state machine of the engineered biological system 100 are verified. To do so, the default state may be verified in block 410 and each functional state may be verified in block 412. In some embodiments, the deactivated state may also be verified in block 414. Typically, the deactivated state may not provide a transition path from the state; however, in some embodiments a one-time transition from the deactivated state may be designed into the engineered biological system 100. To verify each state, a sample transition trigger (e.g., a sample trigger condition) may be exposed to the engineered biological system 100 to cause the system to transition to the various states. In this way, the correction transitioning of the engineered biological system 100 may be verified. Additionally, the various state functions to be performed by the engineered biological system 100 in each of the associated states may be verified in block 416. As with the state transitions, sample functional triggers may be exposed to the engineered biological system 100 to cause the system 100 to perform the associated state function, if required.

After the operation of the engineered biological system 100 has been tested in block 408, it is determined whether the engineered biological system 100 was validated based on such testing. If not, the method 400 loops back to block 402 in which the design of the engineered biological system 100 may be refined or otherwise redesigned. If, however, the operation of the engineered biological system 100 is verified, the method 400 advances to block 420 in which the engineered biological system 100 is activated in the target host. For example, the engineered biological system may be injected, implanted, applied, or otherwise associated with the host and activated in situ. Once activated, the engineered biological system 100 begins in the default state and commences operation as defined by its associated state machine.

It should be appreciated from the state machines 200 and 300 and methods 400 and 500 described above, that the engineered biological system 100 may be designed to perform various functions in various states, which may be transitioned to in response to corresponding trigger conditions. As such, the engineered biological system 100 may be designed to have particular global behaviors and functionality based on its associated state machine mapping. For example, an engineered biological system 100 designed according to the technologies discussed herein may be configured to perform various functions based on established geo-fencing of the host (e.g., the various transition triggers may be based on defined locations within the host, to “keep alive” (e.g., to not transition the deactivated state) based on the presence or absence of other biological system or trigger condition, to send signals or perform other biological functions based on various functional triggers, used to provide a form of authentication based on responses of the engineered biological system 100 to various stimuli, reproduce or replicate based on the presence of various stimuli, provide biological rights management by selectively expressing different versions of genes in response to corresponding stimuli, and/or other designed functions.

It should further be appreciated that the engineered biological system 100 manufactured using the methods 400 and 500 and described in detail above are not naturally occurring biological systems. Rather, the engineered biological system 100 is manufactured, designed, and/or created according to the technologies described herein to perform the various associated functions and exhibit the designed behavior.

### EXAMPLES

Illustrative examples of the devices, systems, and methods disclosed herein are provided below. An embodiment of the devices, systems, and methods may include any one or more, and any combination of, the examples described below.

Example 1 includes an system for performing a biological function, the system comprising an engineered biological system to (i) transition from a default state to a first functional state in response to the presence of a first transition trigger and (ii) perform a first biological function associated with the first functional state while in the first functional state.

Example 2 includes the subject matter of Example 1, and wherein to transition from the default state to the first functional state comprises to transition from the default state to the first functional state in response to the presence of at least one of (i) an energetic transition trigger, (ii) an organic transition trigger, (iii) a chemical transition trigger, (iv) an external transition trigger, (v) a contextual transition trigger, (vi) a proximity transition trigger, or a temporal transition trigger.

Example 3 includes the subject matter of any of Examples 1 and 2, and wherein to perform the first biological function comprises to perform a first biological function associated with the first functional state in response to the presence of the first functional trigger.

Example 4 includes the subject matter of any of Examples 1-3, and wherein the engineered biological system is further to perform a second biological function associated with the first functional state in response the presence of a second functional trigger.

Example 5 includes the subject matter of any of Examples 1-4, and wherein the engineered biological system is further to transition from the first functional state to a second functional state in response to the presence of a second transition trigger.

Example 6 includes the subject matter of any of Examples 1-5, and further wherein the engineered biological system is further to perform a second biological function associated with the second functional state while in the second functional state.

Example 7 includes the subject matter of any of Examples 1-6, and wherein the engineered biological system is further to transition from the first functional state to a deactivated state in response the presence of a second transition trigger, wherein the engineered biological system performs no function while in the deactivated state.

Example 8 includes a method for performing a biological function, the method comprising transitioning, by an engineered biological system, from a default state to a first functional state in response to the presence of a first transition trigger; and performing, by the engineered biological system, a first biological function associated with the first functional state while in the first functional state.

Example 9 includes the subject matter of Example 8, and wherein transitioning from the default state to the first functional state comprises transitioning, by the engineered biological system, from the default state to the first functional state in response to the presence of at least one of (i) an energetic transition trigger, (ii) an organic transition trigger, (iii) a chemical transition trigger, (iv) an external transition trigger, (v) a contextual transition trigger, (vi) a proximity transition trigger, or a temporal transition trigger.

Example 10 includes the subject matter of any of Examples 8 and 9, and wherein performing the first biological function comprises performing, by an engineered biological system, a first biological function associated with the first functional state in response to the presence of the first functional trigger.

Example 11 includes the subject matter of any of Examples 8-10, and further including performing, by the engineered biological system, a second biological function associated with the first functional state in response the presence of a second functional trigger.

Example 12 includes the subject matter of any of Examples 8-11, and further including transitioning, by the engineered biological system, from the first functional state to a second functional state in response to the presence of a second transition trigger.

Example 13 includes the subject matter of any of Examples 8-12, and further including performing, by the engineered biological system, a second biological function associated with the second functional state while in the second functional state.

Example 14 includes the subject matter of any of Examples 8-13, and further including transitioning, by the engineered biological system, from the first functional state to a deactivated state in response the presence of a second transition trigger, wherein the engineered biological system performs no function while in the deactivated state.

Example 15 includes a method for manufacturing an engineered biological system, the method comprising determining a plurality of functions to be performed by the engineered biological system; determining a plurality of states of the engineered biological system, wherein the engineered biological system is to perform at least one function of the plurality of functions in at least one state of the plurality of states; determining at least one state transition between two states of the plurality of states; determining a biological key for each state transition, wherein the biological key causes the engineered biological system to transition from a first state to a second state defined by an associated state transition and in response to presence of a transition trigger corresponding to the biological key; and generating a state machine mapping for the engineered biological system based on the determined states, state transitions, and biological keys.

Example 16 includes the subject matter of Example 15, and wherein determining the plurality of functions comprises determining a plurality of biological functions of the engineered biological system.

Example 17 includes the subject matter of any of Examples 15 and 16, and further including a determining a function trigger for at least one function of the plurality of functions, wherein presence of the function trigger causes the engineered biological system to perform the associated at least one function.

Example 18 includes the subject matter of any of Examples 15-17, and wherein determining the plurality of states of the engineered biological system comprises determining a default state of the biological system, wherein the engineered biological system is configured to begin in the default state upon activation in a host.

Example 19 includes the subject matter of any of Examples 15-18, and wherein determining the plurality of functions comprises determining a default function to be performed by the engineered biological system while in the default state.

Example 20 includes the subject matter of any of Examples 15-19, and further including a determining a default function trigger for the default function, wherein the presence of the default function trigger causes the engineered biological system to perform the associated default function.

Example 21 includes the subject matter of any of Examples 15-20, and wherein determining the plurality of states of the engineered biological system comprises determining a deactivated state of the biological system, wherein the engineered biological system is configured to transition to the deactivated state in response to the presence of deactivated state transition trigger.

Example 22 includes the subject matter of any of Examples 15-21, and wherein determining the plurality of states of the engineered biological system comprises determining a plurality of functional states, wherein the engineered biological system is to perform at least one function of the plurality of functions in each functional state.

Example 23 includes the subject matter of any of Examples 15-22, and wherein determining at least one state transition comprises determining a first state transition between a first functional state and a second functional state of the plurality of functional states, and determining a biological key comprises determining a first biological key for the first state transition, wherein the engineered biological system is to transition from the first functional state to the second function state in response to activation the presence of the first transition trigger.

Example 24 includes the subject matter of any of Examples 15-23, and wherein each biological key comprises at least one of: (i) an energetic key that causes the engineered biological system to transition states in response to a corresponding energetic transition trigger, (ii) an organic key having a that causes the engineered biological system to transition states in response to an organic transition trigger, (iii) a chemical key that causes the engineered biological system to transition states in response to a chemical transition trigger, (iv) an external key that causes the engineered biological system to transition states in response to a transition trigger external from the host, (v) a contextual key that causes the engineered bio-system to transition states in response to a contextual transition trigger indicative of a context of the engineered biological system, or (vi) a proximity key that causes the engineered biological system to transition states in response to a proximity transition trigger indicative of the engineered biological system being in proximity to a target system.

Example 25 includes the subject matter of any of Examples 15-24, and wherein the transition trigger comprises at least one of (i) an energetic transition trigger, (ii) an organic transition trigger, (iii) a chemical transition trigger, (iv) an external transition trigger, (v) a contextual transition trigger, (vi) a proximity transition trigger, or a temporal transition trigger.

Example 26 includes the subject matter of any of Examples 15-25, and further including manufacturing the engineered biological system based on the state machine mapping; and verifying operation of the engineered biological system in a quarantine environment.

Example 27 includes the subject matter of any of Examples 15-26, and wherein verifying operation of the engineered biological system comprise verifying each state transition of the state machine mapping of the engineered biological system.

Example 28 includes the subject matter of any of Examples 15-27, and wherein verifying operation of the engineered biological system comprise verifying each function the engineered biological system.

Example 29 includes the subject matter of any of Examples 15-28, and further including activating the engineered biological system in a host in response to verifying operation of the biological system in the quarantine environment.

Example 30 includes the subject matter of any of Examples 15-29, and wherein activating the engineered biological system comprises transitioning, by the engineered biological system, from a default state of the plurality of states to a first functional state of the plurality of states in response the presence of a corresponding transition trigger, wherein the engineered biological system is to perform a first function of the plurality of functions in the first functional state.

Example 31 includes one or more computer-readable storage media comprising a plurality of instructions stored thereon that, in response to execution, cause a computing device to perform the method of any of Examples 15-30.

Example 32 includes an engineered biological fabrication system, the system comprising means for performing the method of any of Examples 15-30.

1. An system for performing a biological function, the system comprising:
an engineered biological system to (i) transition from a default state to a first functional state in response to the presence of a first transition trigger and (ii) perform a first biological function associated with the first functional state while in the first functional state.

2. The system of claim 1, wherein to transition from the default state to the first functional state comprises to transition from the default state to the first functional state in response to the presence of at least one of (i) an energetic transition trigger, (ii) an organic transition trigger, (iii) a chemical transition trigger, (iv) an external transition trigger, (v) a contextual transition trigger, (vi) a proximity transition trigger, or a temporal transition trigger.

3. The system of claim 1, wherein to perform the first biological function comprises to perform a first biological function associated with the first functional state in response to the presence of the first functional trigger.

4. The system of claim 3, wherein the engineered biological system is further to perform a second biological function associated with the first functional state in response the presence of a second functional trigger.

5. The system of claim 1, wherein the engineered biological system is further to transition from the first functional state to a second functional state in response to the presence of a second transition trigger.

6. The system of claim 5, further wherein the engineered biological system is further to perform a second biological function associated with the second functional state while in the second functional state.

7. The system of claim 1, wherein the engineered biological system is further to transition from the first functional state to a deactivated state in response the presence of a second transition trigger, wherein the engineered biological system performs no function while in the deactivated state.

8. A method for performing a biological function, the method comprising:
transitioning, by an engineered biological system, from a default state to a first functional state in response to the presence of a first transition trigger; and
performing, by the engineered biological system, a first biological function associated with the first functional state while in the first functional state.

9. The method of claim 8, wherein transitioning from the default state to the first functional state comprises transitioning, by the engineered biological system, from the default state to the first functional state in response to the presence of at least one of (i) an energetic transition trigger, (ii) an organic transition trigger, (iii) a chemical transition trigger, (iv) an external transition trigger, (v) a contextual transition trigger, (vi) a proximity transition trigger, or a temporal transition trigger.

10. The method of claim 8, wherein performing the first biological function comprises performing, by an engineered biological system, a first biological function associated with the first functional state in response to the presence of the first functional trigger.

11. The method of claim 10, further comprising performing, by the engineered biological system, a second biological function associated with the first functional state in response the presence of a second functional trigger.

12. The method of claim 18, further comprising transitioning, by the engineered biological system, from the first functional state to a second functional state in response to the presence of a second transition trigger.

13. The method of claim 12, further comprising performing, by the engineered biological system, a second biological function associated with the second functional state while in the second functional state.

14. The method of claim 8, further comprising transitioning, by the engineered biological system, from the first functional state to a deactivated state in response the presence of a second transition trigger, wherein the engineered biological system performs no function while in the deactivated state.

15. A method for manufacturing an engineered biological system, the method comprising:
determining a plurality of functions to be performed by the engineered biological system;
determining a plurality of states of the engineered biological system, wherein the engineered biological system is to perform at least one function of the plurality of functions in at least one state of the plurality of states;
determining at least one state transition between two states of the plurality of states;
determining a biological key for each state transition, wherein the biological key causes the engineered biological system to transition from a first state to a second state defined by an associated state transition and in response to presence of a transition trigger corresponding to the biological key; and
generating a state machine mapping for the engineered biological system based on the determined states, state transitions, and biological keys.

16. The method of claim 15, wherein determining the plurality of functions comprises determining a plurality of biological functions of the engineered biological system.

17. The method of claim 15, further comprising a determining a function trigger for at least one function of the plurality of functions, wherein presence of the function trigger causes the engineered biological system to perform the associated at least one function.

18. The method of claim 15, wherein determining the plurality of states of the engineered biological system comprises determining a default state of the biological system, wherein the engineered biological system is configured to begin in the default state upon activation in a host,
wherein determining the plurality of functions comprises determining a default function to be performed by the engineered biological system while in the default state, and
further comprising determining a default function trigger for the default function, wherein the presence of the default function trigger causes the engineered biological system to perform the associated default function.

19. The method of claim 15, wherein determining the plurality of states of the engineered biological system comprises determining a deactivated state of the biological system, wherein the engineered biological system is configured to transition to the deactivated state in response to the presence of deactivated state transition trigger.

20. The method of claim 15, wherein determining the plurality of states of the engineered biological system comprises determining a plurality of functional states, wherein the engineered biological system is to perform at least one function of the plurality of functions in each functional state.

21. The method of claim 20, wherein:
determining at least one state transition comprises determining a first state transition between a first functional state and a second functional state of the plurality of functional states, and
determining a biological key comprises determining a first biological key for the first state transition, wherein the engineered biological system is to transition from the first functional state to the second function state in response to activation the presence of the first transition trigger.

22. The method of claim 15, further comprising:
manufacturing the engineered biological system based on the state machine mapping; and
verifying operation of the engineered biological system in a quarantine environment.

23. The method of claim 22, wherein verifying operation of the engineered biological system comprise verifying each function the engineered biological system.

24. The method of claim 22, further comprising activating the engineered biological system in a host in response to verifying operation of the biological system in the quarantine environment.

25. The method of claim 24, wherein activating the engineered biological system comprises transitioning, by the engineered biological system, from a default state of the plurality of states to a first functional state of the plurality of states in response the presence of a corresponding transition trigger, wherein the engineered biological system is to perform a first function of the plurality of functions in the first functional state.
